SEQUENCE LISTING (10 sequences)

SEQ ID NO: 1 - DNA, 24 bp, Artificial Sequence
  Chemically synthesized
gagaactcctgactgaactggacg

SEQ ID NO: 2 - DNA, 22 bp, Artificial Sequence
  Chemically synthesized
ccatattccctgctgagcgtag

SEQ ID NO: 3 - DNA, 21 bp, Artificial Sequence
  Chemically synthesized
aactgaatcgcctccaagcag

SEQ ID NO: 4 - DNA, 21 bp, Artificial Sequence
  Chemically synthesized
cgaactggcgagaactggatg

SEQ ID NO: 5 - DNA, 21 bp, Artificial Sequence
  Chemically synthesized
cctgctcatcagcaagattcg

SEQ ID NO: 6 - DNA, 21 bp, Artificial Sequence
  Chemically synthesized
gtggtgagcttaagggtacgg

SEQ ID NO: 7 - DNA, 21 bp, Artificial Sequence
  Chemically synthesized
gctttccctctgttgacccac

SEQ ID NO: 8 - DNA, 21 bp, Artificial Sequence
  Chemically synthesized
agatgttgagggcagctcgat

SEQ ID NO: 9 - DNA, 21 bp, Artificial Sequence
  Chemically synthesized
ctgaaggtcaaagggaatgtg

SEQ ID NO: 10 - DNA, 21 bp, Artificial Sequence
  Chemically synthesized
ggacagagtcttgatgatctc

(19) United States
(12) Patent Application Publication
(10) Pub. No.: US 2004/0214767 A1
(43) Pub. Date: Oct. 28, 2004

(54) Regulation of follicular development and ovulation by cytoskeletal genes and gene products

(76) Inventors: Tae H. Ji, Lexington, KY (US); Inhae Ji, Lexington, KY (US)

Correspondence Address: McDERMOTT, WILL & EMERY, 600 13th Street, N.W., Washington, DC, 20005-3096, US

(21) Appl. No.: 10/696,562

(22) Filed: Oct. 30, 2003

Related U.S. Application Data
(60) Provisional application No. 60/422,097, filed on Oct. 30, 2002.

(51) Int. Cl. A61K 38/24

(52) U.S. Cl. 514/12

(57) ABSTRACT

Methods for altering the regulation of follicular development and ovulation by modulating cytoskeletal genes and gene products are disclosed. More particularly, β-tubulin, tropomyosin-4 and kinesin heavy chain are used to control follicular development and/or the ovulation cycle.

This application claims priority to U.S. provisional application Serial No. 60/422,097, filed Oct. 30, 2002, incorporated herein by reference.

### FIELD OF THE INVENTION

This invention relates to the regulation of follicular development and ovulation by modulating cytoskeletal genes and gene products. More particularly, this invention relates to the use of β-tubulin, tropomyosin-4 and kinesin heavy chain to control follicular development and/or the ovulation cycle.

### BACKGROUND OF THE INVENTION

The follicle of the mammalian ovary develops in several distinct stages during folliculogenesis. Primordial follicles are recruited from the resting pool to develop towards the preovulatory stage. The critical factors controlling the initial recruitment of primordial follicles are poorly understood (McGee et al., 2000 Endocr Rev 21:200-214). Follicular development through the primary, secondary, antral and eventually preovulatory stages is controlled by both endocrine and paracrine factors (McGee et al., Endocr Rev 21:200-214; Hirshfield, Int Rev Cytol 124:43-101; Richards et al., 1995 Recent Prog Horm Res 50:223-254; Rannikki et al., 1995 Mol Cell Endocrinol 107:199-208; and Gougeon A., Endocr Rev 17:121-155).

Primordial and primary follicles have an oocyte surrounded by a single layer of granulosa cells. Secondary, antral and preovulatory follicles consist of several layers of granulosa cells and an outer layer of thecal cells. Granulosa cells are the nurse cells to the oocyte and are the only cells in the follicle able to respond to follicle stimulating hormone (FSH). Functional FSH receptors are found by day seven in rats, when well developed preantral follicles can be detected (Rannikki et al., 1995 Mol Cell Endocrinol. 107:199-208). While follicles at this stage can respond to FSH, they are not absolutely dependent on its presence. For example, follicles of hypophysectomized and GnRH agonist-treated juvenile rats can progress to the antral stage though at a slower pace and in fewer numbers (McGee et al., 1997 Biol Reprod 57:990-998). Thus, the development of early follicles is probably under the influence of, but not dependent on FSH. In contrast, FSH as well as LH is crucial for proper follicle development past the antral stage. In fact, FSH is the predominant survival factor at this point (Chun et al., 1996 Endocrinology 137:1447-1456).

The fate of granulosa cells at each stage of follicular development is determined in part by hormone regulated gene expression (Richards JS. 1994 Endocrine Review 15:725-751). In addition, the response of granulosa cells to FSH is developmental stage dependent. For example, at early stages of follicular growth, FSH supports mitotic activity while a different and specific set of genes is induced by FSH during the final stages of preovulatory development (Richards JS 1980 Physiol Rev 60:51-89; and Hirshfield AN 1986 Patterns of [3H] thymidine incorporation). A number of genes regulated by FSH during the later stages of follicle development, i.e., the preovulatory follicle, have been identified and characterized (Richards JS. 1994 Hormonal control of gene expression in the ovary. Endocrine Review 15:725-751). However, only a few genes have been identified that are regulated by FSH during the early stages of development. These include inhibin alpha (Feng et al., 1989 Mol Endocrinol 3:1914-1925; and et al., 1991 Mol Endocrinol 5:521-534), Wilms' tumor gene (Hsu et al., 1995 Mol Endocrinol 9:1356-1366) and cyclin D2 (Sicinski et al., 1996 Nature 384:470-4744). One of the major landmarks for folliculogenesis is formation of the antrum, which likely involves changes in the cytoskeleton. However, due to a lack of a suitable experimental system, little is known about the expression of cytoskeletal genes during follicular development, except that actin, the primary component of actin cytoskeleton, is constantly expressed.

Rat ovarian granulosa (ROG) cells are a homogeneous clonal cell line established from immature granulosa cells of the rat ovary (Li R, et al., 1997 Follicle-stimulating hormone induces terminal differentiation in a predifferentiated rat granulosa cell line (ROG). Endocrinology 138:2648-2657). These cells grow in a serum free-defined medium containing activin A, but not FSH. They maintain many characteristics of undifferentiated immature cells, i.e., lack of steroidogenesis and ability to respond to luteinizing hormone (LH). Upon exposure to FSH, the cells become post-mitotic and highly steriodogenic, similar to mature granulosa cells of a dominant follicle. FSH-stimulated ROG cells also become dependant on the continued presence of FSH and will undergo apoptosis upon its removal (Li R, et al. 1997). In addition, ROG cells form a structure resembling a follicle when cultured in the presence of an oocyte/cumulus cell complex (Li et al., 1997 Endocrinology 138:4477-448016). The present inventors have previously shown that in response to FSH, ROG cells undergo massive actin cytoskeleton rearrangements within three hours, leading to changes in cell-cell interactions (Grieshaber et al., 2000, Endocrinology 141:3461-3470). However, there remains a need to better understand expression of the genes involved in follicular development and ovulation in order to manipulate the regulation of these genes.

### SUMMARY OF THE INVENTION

In one aspect of the present invention there is provided a method of regulating follicular development and/or ovulation in a patient by modulating the genes and gene products of β-tubulin, tropomyosin-4, kinesin heavy chain or a combination thereof.

In another aspect of the invention there is provided a method of altering regulation of follicular development, atrophy and/or ovulation comprising exposing immature granulosa cells to an effective amount of follicle stimulating hormone at an early stage of granulosa cell differentiation.

In yet another aspect of the invention there is provided a method of regulating differentiation of immature granulosa cells comprising exposing the immature granulosa cells to an effective amount of follicle stimulating hormone at an early stage of granulosa cell differentiation.

The invention also provides a method of altering beta-tubulin gene expression during follicular development comprising exposing the large preantral and early antral follicules of an animal to an effective amount of follicle stimulating hormone at an early stage of immature granulosa cell differentiation.

### BRIEF DESCRIPTION OF THE DRAWINGS

FIG. 1. Differential display autoradiogram. ROG cells were cultured for 6 hours in the absence (−) or presence (+) of FSH, and total RNA was extracted and subjected to differential display after RT-PCR (A) on DNA sequencing gels as described in the Examples. (B) A portion of the gels shows bands that responded to FSH. The white arrow indicates the band with a decrease in the intensity upon FSH treatment and black arrow represents the band with increased intensity. (C) FSH responsive bands were excised, PCR-amplified and electrophoresed. The DNAs were isolated and cloned.

FIG. 2. Temporal expression of mRNAs in ROG cells. The cDNA fragment in each of the clones described in the FIG. 1 legend was amplified and slot-blotted. These blots were probed with [gamma-³²P]dCTP-labeled probes, which were prepared as follows: ROG cells were cultured for 0 hours, 6 hours and 24 hours in the presence of 30 ng/ml FSH, total RNA was isolated, and reverse transcribed to produce [alpha-³²P]dCTP-labeled cDNA probes, as described in the Examples. The experiments were repeated several times, and 14 clones showed consistent patterns of expression. They were grouped according to their temporal expression pattern. (A) Six clones were up-regulated by FSH treatment (JK#23-chloride channel protein, JK#26-unknown, JK#28-unknown, JK#56-inhibin alpha subunit, JK#78-unknown, JK#27-unknown,). (B) Two clones showed down-regulation (JK#24-3 alpha hydroxysteroid dehydrogenase, JK#39-unknown). (C) Six clones were first up-regulated and then, down-regulation (JK#7-tropomyosin-4, JK#10-unknown, JK#18-SKB1H, JK#25-kinesin heavy chain, JK#34-unknown, JK#46-beta-tubulin). The identities of the clones are summarized in Table 1.

FIG. 3. Northern blot of temporal cytoskeletal gene mRNA in ROG cells. ROG cells were cultured in the presence of 30 ng/ml FSH for 0, 6 and 24 h, and total RNA was isolated. The mRNA expression level was analyzed by Northern blotting. Total RNA (10 μg) was loaded in each lane, electrophoresed, visualized with ethidium bromide, blotted and probed for TM4, Khc, beta-tubulin or inhibin alphasubunit. The 28S rRNA bands of all gel lanes that were stained with ethidium bromide are shown in the lower panel. The size markers (0.78-4.40 kb RNA ladder, Life Technologies, Inc.) are shown on the left. Autoradiograms show one respective band by TM4 (0.9 kb), Khc (2.7 kb), and inhibin alphasubunit (1.6 kb) cDNA probes, respectively, and three bands with beta-tubulin probe.

FIG. 4. Fluorescent microscopy of ROG cells stained for beta-tubulin and f-actin proteins. ROG cells were grown on poly-D-Lysine/fibronectin coated 12 mm number 1 glass coverslips in defined culture media in the absence of FSH (A and D) or in the presence of 30 ng/ml FSH (B, C, E and F) for 3 hours (B and E) or 24 h (C and F). The cells were stained for f-actin (A, B and C) or beta-tubulin (D, E and F), and examined under a fluorescence confocal microscope. Arrows and arrowheads indicate lamellipodia and filopodia, respectively. Note the similar level of fluorescence signal for f-actin (A-C) and the increased signal for beta-tubulin protein among the treatment groups (D-F). The scale bar represents 15 μm.

FIG. 5. Temporal expression of beta-tubulin mRNA during follicular development. Immature 22-23 day old rats were injected with PMSG (15 IU) and kept for 0, 3, 6, 24 or 48 hours. Rats primed with PMSG for 48 hours were additionally injected with hCG (10 IU) and kept for 3, 6 or 24 hours. Ovaries were excised and sectioned. Ovarian sections were hybridized with [⁵S]-UTP-labeled beta-tubulin (left panels) or inhibin a (right panels) antisense probe. Bright field images (hematoxylene stainig) are aligned in the right side of the corresponding dark field images. PAF, preantral follicle; EAF, early antral follicle; LAF, large antral follicle; POF, preovulatory follicle. Magnification is ×100.

FIG. 6. Cell-type specific expression of β-tubulin mRNA. Ovarian sections of rats primed with PMSG for 6 hours were hybridized with the β-tubulin riboprobe. The slide was examined for dark field images (upper) and bright field images (lower). An, antrum; PAF, preantral follicle; In, interstitial cells; GC, granulosa cells; Th, theca cells. Photographs are taken at X100 magnification for A-D and X400 for E and F.

FIG. 7. Temporal expression of cytoskeletal genes in primary granulosa cells. Rat granulosa cells were isolated from the ovaries of 17□-estradiol primed, 24 day old immature rats described in the Examples. They were cultured in the presence of 30 ng FSH/ml for up to 48 hours. Total RNA was isolated, amplified for semiquantitative RT-PCR for TM4, Khc, beta-tubulin or inhibin alpha mRNA, resolved on 2% agarose gel, stained with SYBR® Green I (Molecular Probes), and phosphoimaged as described in the Examples. Ribosomal protein L-19 mRNA was used for internal control. Values are the meanfold increase over control (0 h)±SE. Values were pooled from two separate experiments.

FIG. 8. Effects of CHX and alpha-amanitin treatment on the FSH/forskolin-induced expression of the cytoskeletal genes. Granulosa cells were isolated from the ovaries of 170 estradiol primed 24 day old immature rats. They were cultured in the medium containing FSH (30 ng/ml) or forskolin (10 microM, FSK) in the presence or absence of CHX (10 μg/ml) for 6 hours (A) or 24 hours (B). In addition, the 24 hours cells were also cultured, likewise, in the presence of alpha-amanitin (30 microg/ml, AMA). Total RNA was isolated and analyzed for mRNAs of TM4, Khc, beta-tubulin and inhibin alpha as described in the description of FIG. 7. Values are the meanfold increase over control (no treatment)±SE. Values were pooled from two separate experiments.

### DETAILED DESCRIPTION OF THE INVENTION

To identify cytoskeleton and other genes regulated by FSH during immature granulosa cell differentiation, the present inventors performed differential display of mRNA on ROG cells.

This method has been successfully used to identify genes involved in apoptosis in ovaries after estrogen withdrawal (Svanberg B, et al.,1999 J Endocrinol 163:309-316), in FSH regulation of pig ovary granulosa cells (Clouscard-Martinato et al., 1998 Anim Genet 29:98-106) and in hCG regulation of 3alpha-hydroxysteroid dehydrogenase mRNA expression (Espey et al., 2001 Biology of Reproduction 65:72-78).

Eighty genes, including three cytoskeletal genes, class I β-tubulin, tropomyosin-4 and kinesin heavy chain, were identified using differential display of ROG cells. The FSH dependent mRNA and protein expression of these identified genes in ROG cells, primary granulosa cells and in vivo is described herein.

Differential Display of FSH Responsive Genes

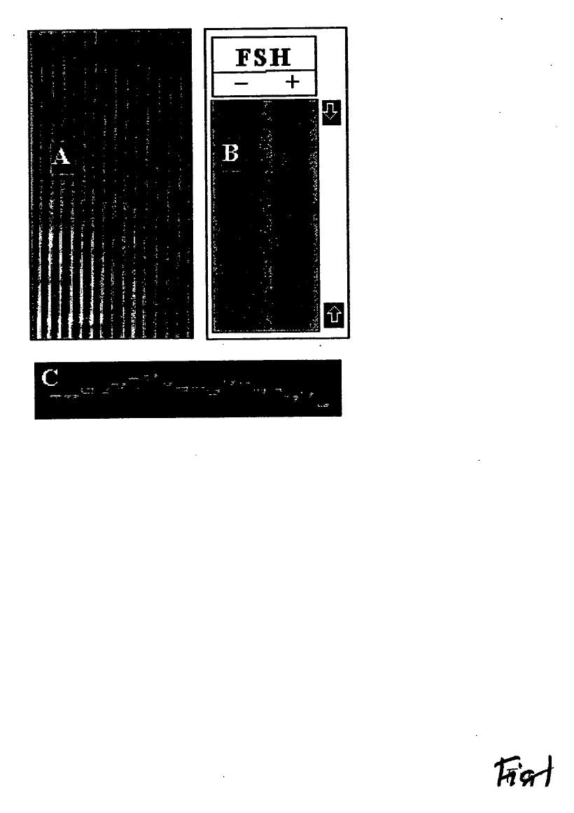

ROG cells are a clonal cell line derived from a P14 immature rat and thus, undifferentiated. These cells grow in a defined medium containing activin A but free of serum and FSH. They express the FSH receptor and respond to exogenous FSH with a burst of proliferation, becoming both steriodogenic and terminally differentiated by ˜96 hours post stimulation. The present inventors have previously shown that ROG cells undergo rapid morphological changes, including formation of lamellipodia and filopodia within three hours of FSH treatment (Grieshaber et al., 2000 Endocrinology 141:3461-3470). With these considerations in mind, FSH responsive genes in granulosa cells early in the path to terminal differentiation were sought. ROG cells were cultured in the absence or presence of 30 ng/ml FSH for six hours, total RNA was extracted, and differential display was performed (FIG. 1A). Differentially expressed bands (FIG. 1B) were excised, PCR-amplified and electrophoresed. Electrophoresis of amplified samples revealed 80 sets of differential bands (an example of which can be seen in FIG. 1C), of which 54 were up-regulated and 26 were down-regulated.

Identification and Characteristics of FSH Responsive Genes

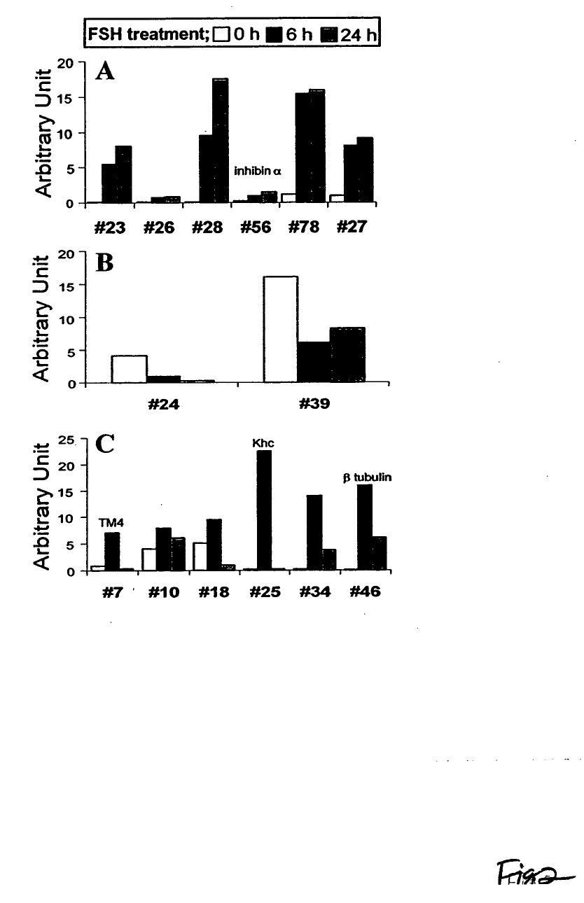

The cDNAs were extracted from the gels, cloned and sequenced. Sequence comparison revealed 61 unique cDNAs and 19 duplicates. To verify the time-dependent FSH-responsiveness of the unique cDNAs, they were slot-blotted and probed with [alpha-³²P]dCTP-cDNA probes. To prepare these probes, mRNA extracted from ROG cells treated with FSH for 0, 6 and 24 hours were reverse-transcribed with [alpha-³²P]dCTP. Repeated slot blot analyses revealed 14 genes (FIG. 2), whose mRNA expression levels changed consistently and significantly over a 24 h period after FSH treatment. Interestingly, their gene expression was diverse. Some were either up-regulated (FIG. 2A) or down-regulated (FIG. 2B), whereas others were initially up-regulated and subsequently, down-regulated (FIG. 2C). Three of these genes encode cytoskeletal proteins.

Clone #24 is 3 alpha-hydroxysteroid dehydrogenase and is down-regulated. This gene and the gene products may be involved in steroid production, in particular estrogen synthesis.

FSH Responsive Cytoskeletal Genes in ROG Cells

A Blast search revealed that clone JK#7 was 100% homologous to rat tropomyosin-4 (TM4; GeneBank No. Y00169), JK#25 was 100% homologous to a rat EST clone (GeneBank No. AI137325) and 98% homologous to mouse kinesin heavy chain (Khc; GeneBank No. L27153), and JK#46 was 100% homologous to rat class I β-tubulin (GeneBank No. ABO 11679). It should be noted that all three genes were up-regulated at six hours post FSH treatment and subsequently, down-regulated by 24 hours (FIG. 2C). Clone #56 was inhibin alpha, and its expression was induced and sustained by FSH (FIG. 2A). This is consistent with previous reports that the gene is induced and sustained by FSH in immature granulosa cells (27,28). Therefore, it has been routinely used as a control and representative FSH-regulated gene (29,30).

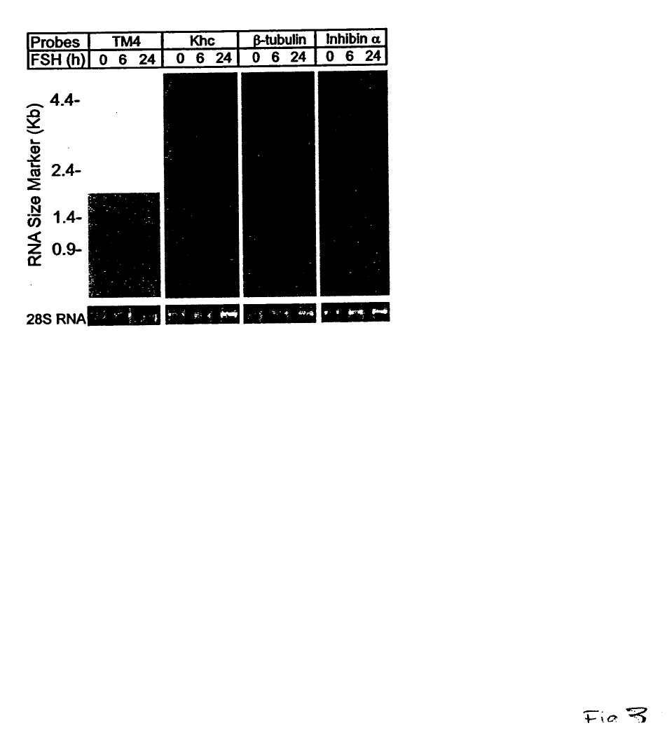

Whether the changes in the three gene transcripts shown in FIG. 2 indeed reflect their mRNA levels or putative experimental variations, in particular PCR dependent amplification, was analyzed by Northern blot analysis. Northern blots, however, require substantial amounts of mRNAs. For that reason, a large quantity of ROG cells were grown and exposed to FSH for 0, 6 or 24 hours. Total RNA was extracted from these cells and the individual transcripts were directly measured by Northern blot. An equal amount of total RNA was applied to individual sample lanes. The large and small rRNA bands and tRNA band appeared distinctly and their respective band intensities were invariable in the gel lanes as seen in the similar intensity of the 28S rRNA bands in all sample lanes (FIG. 3), suggesting the integrity of the RNA samples. Inhibin alpha mRNA was also examined as a control. The results confirmed the FSH— and time-dependent expression of the three cytoskeletal genes in ROG cells (FIG. 3).

Autoradiograms revealed a 0.9 kb band for TM4, 2.7 kb band for Khc, and 1.6 kb band for the inhibin alphasubunit. In contrast to the single band of TM4, Khc and inhibin alpha mRNAs, beta-tubulin mRNAs showed three isoforms of 2.3, 3.1, and 4.4 kb bands. There are striking differences in the mRNA expression of these genes. TM4 and Khc mRNAs were not detectable before FSH treatment, dramatically increased after FSH treatment for six hours, and then decreased to background level by 24 hours, consistent with results shown in FIG. 2. It is interesting that beta-tubulin mRNA shows three isoforms, which diversely responded to FSH. The largest 4.4 kb band of beta-tubulin mRNA markedly increased at six hours of FSH treatment and then, disappeared by 24 hours. In contrast, the 2.3 and 3.1 kb mRNA bands increased at six hours and sustained their intensity at 24 hours. Therefore, the total intensity of the three beta-tubulin mRNA bands markedly increased at six hours and partially decreased at 24 hours, consistent with the observation in FIG. 2. This FSH-dependent differential expression of beta-tubulin mRNA is novel and raises an intriguing question on the function and regulation of the three mRNA isoforms and their role in granulosa cell differentiation. Unlike these cytoskeletal genes, inhibin alpha mRNA gradually increased throughout FSH treatment, which is consistent with previous reports.

Expression and Localization of β-Tubulin and Tropomyosin Proteins

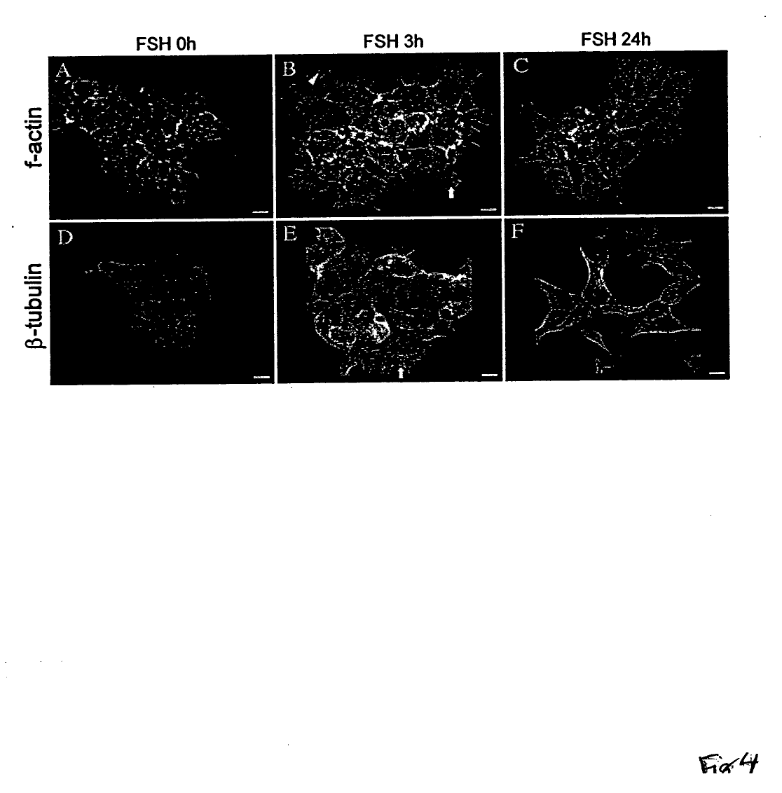

To determine whether the changes in the mRNA expression are reflected in the protein expression and cell morphology, ROG cells were cultured in the presence or absence of 30 ng/ml FSH and immunostained with anti-β-tubulin antibody or anti-tropomyosin antibody. The cells were also stained with anti-f-actin antibody as a comparison. Staining of FSH-stimulated ROG cells with anti-actin antibody shows the formation of lamellipodia and filopodia as early as three hours (FIG. 4). By 24 hours, the cells around the periphery of the cell clump had spread to form a monolayer, consistent with the inventors' previous observations (Grieshaber et al., 2000 Endocrinology 141:3461-3470). Although β-tubulin staining was light in untreated samples (FIG. 4D), it was intense and distributed throughout the cells by three hours after FSH treatment (FIG. 4E). The results were the same for six hours treatment (data not included). These observations suggest that β-tubulin was produced en mass and assembled into an extensive network of microtubules. By 24 hours, β-tubulin staining was reduced (FIG. 4F). These results show that beta-tubulin appeared in parallel to the onset of its FSH-dependent mRNA synthesis but disappeared slower than its mRNA.

Kinesin is associated with microtubules but in a quantity significantly less than tubulin. The distribution of tropomyosin in the cell was similar to that of β-tubulin but not as dramatic (data not included). In contrast to the dramatic increase in the β-tubulin and tropomyosin concentrations, the f-actin concentration did not change in response to FSH but there was massive reorganization of the actin cytoskeleton (FIGS. 4A-C). This result is consistent with the inventors' previous observation that the actin mRNA level was constant regardless of FSH treatment (data not shown). Clearly, the organization and regulation of the microtubules differ from those of the actin cyctoskeletons including lamellipodia and filopodia in ROG cells.

mRNA Expression During Follicular Development

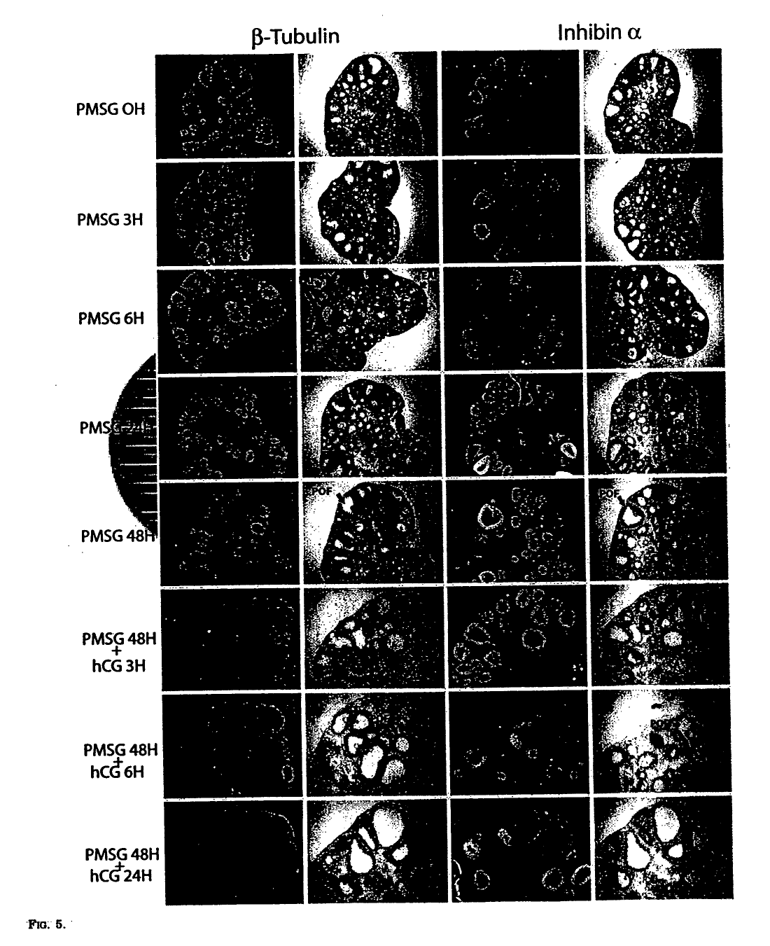

The physiological relevancy of the three cytoskeletal genes to granulosa cell differentiation in vivo was analyzed. To localize the cytoskeletal mRNAs in the ovary and follicle in vivo, sections of an adult rat ovary were in situ hybridized. The β-tubulin and TM4 mRNAs were detected in a few large preantral and early antral follicles (FIG. 5), suggesting that their expression is dependent on follicular development. On the other hand, Khc mRNA expression was not detected, probably due to the relatively small size (160 bp) of the Khc riboprobe (data not shown).

Whether β-tubulin expression is dependent on granulosa cell differentiation and follicular development was also analyzed. Ovaries were excised from immature female rats that were treated with PMSG for 0, 3, 6, 24 or 48 hours (FIG. 5). In addition, those primed with PMSG for 48 hours were additionally treated with hCG for three hours, six hours or 24 hours to promote development of preovulatory follicles. β-tubulin mRNA was prominently detected during the early stages of PMSG priming (for example, PMSG priming for six hours—24 hours), and the labeling intensity was dependent on the follicular development. The large preantran and early antral follicles were major sites of the β-tubulin mRNA expression. Most of the primary follicles were labeled but the level was moderate. In contrast, the large preovulatory follicles of the ovaries primed with PMSG for 48 hours showed either no signal or a markedly lower level of signal. Particularly, it is striking to find that the additional treatment with hCG completely abolished β-tublin mRNA expression in the preovulatory follicle. The β-tubulin sense probe did not label, showing the specificity of the labeling (data not included). These results clearly show that β-tubulin mRNA expression is dependent on the stage of follicular development.

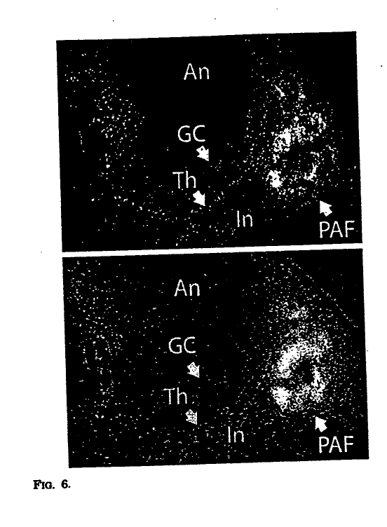

Since inhibin alpha has been proven to be regulated by FSH and used as a FSH-regulated marker gene, it was desirable to compare the expression profiles of β-tubulin and inhibin alpha. For this purpose, tandem sections of the ovary were hybridized with inhibin alpha and β-tubulin riboprobes. The expression patterns of the two gene transcripts were similar up to the early antral stage (FIG. 5). However, in preovulatory follicles inhibin alpha mRNA expression was very high in contrast to suppressed β-tubulin mRNA (FIG. 5, PMSG 48H). A moderate level of β-tubulin mRNA was seen in theca and interstitial cells and the expression persisted in the large antral follicle, even after expression in granulosa cell was diminished (FIG. 6). It should be noted that the basal beta-tubulin mRNA level was readily detectable in granulosa cells, but not in ROG cells. The difference may be attributed to the likely exposure of granulosa cells to FSH before excision from the rat ovaries, whereas ROG cells were not exposed at all.

Expression of the Cytoskeletal Genes in Rat Primary Granulosa Cell Cultures

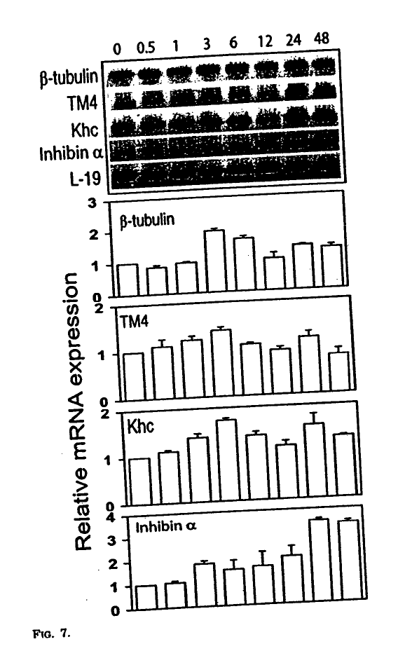

Follicular development requires differentiation of granulosa cells, which is dependent on FSH. Therefore, the association of cytoskeletal gene expression with FSH-dependent differentiation of granulosa cells was analyzed. Immature 21 days old rats were injected with 17-□estradiol for three consecutive days. Granulosa cells were isolated, cultured overnight and treated with FSH for increasing time periods from 0 to 48 hours. This culture system has successfully been used for the studies on FSH-responsive genes. The expression levels of TM4, Khc and beta-tubulin increased to peak at three hours, subsequently decreased to a trough at 12 hours, and increased again to reach another peak at 24 hours (FIG. 7). The level of the internal control, L-19, did not change, whereas inhibin alpha gradually increased to a peak at 24 hours—48 hours. The trend of the three cytoskeletal gene transcripts to peak several hours after FSH treatment is consistent both in ROG cells and in primary granulose cell cultures. In contrast, inhibin alpha mRNA levels continuously increased in both ROG cells and primary culture. These results underscore the similarity in the gene expression profiles of these two cell types.

Different Mechanisms of Cytoskeletal Gene Expressions in Granulosa Cells

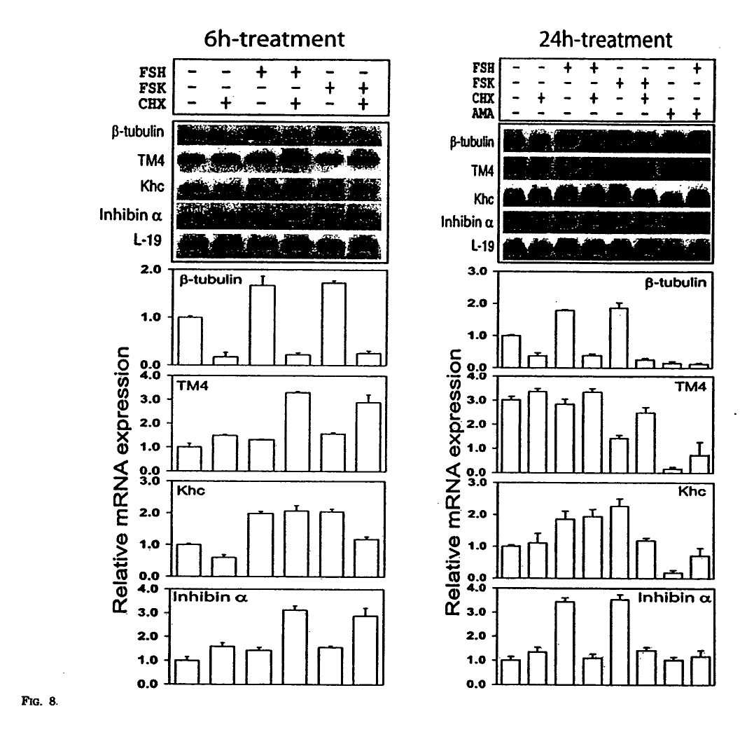

As a step to determine the mechanisms of the FSH-responsive mRNA expression, the granulosa cells were treated with FSH in the presence or absence of a translation inhibitor, CHX, and a transcription inhibitor, alpha amanitin. As shown in FIG. 8, treatment of FSH for six hours enhanced the expression level of β-tubulin, consistent with the result seen in FIG. 7. It was striking to find that β-tubulin mRNA could not be detected when the cells were exposed to CHX, even in the presence of FSH. In addition, the β-tubulin mRNA level decreased to apparent basal level in response to 24 hours exposure to FSH in the presence of CHX (FIG. 8). These results suggest that the de novo synthesis of a protein(s) is necessary to sustain the β-tubulin mRNA level in the absence of FSH and increase its level in response to FSH. In addition, the transcription of the β-tubulin gene appears to be involved in the process, as alpha amanitin blocked both the maintenance and FSH dependent increase of 1-tubulin mRNA. Taken together, these results suggest the interesting possibility that the FSH dependent transcription of 1-tubulin mRNA requires de novo synthesis of a protein factor(s).

It was surprising to find that the effects of CHX on the expression of the three genes were not the same. CHX lowered the Khc mRNA level, though not completely, in cells treated with FSH for six hours, suggesting the existence of CHX insensitive mRNA. This differs from the β-tubulin mRNA. In cells treated with FSH for 24 hours, the effects of CHX and alpha amanitin were marginal. In contrast to the CHX dependent decrease in the levels of the β-tubulin and Khc mRNAs, CHX increased the TM4 mRNA level of the cells treated with FSH for six hours (FIG. 8). This suggests that the FSH dependent increase in TM4 mRNA levels is down regulated by a protein factor(s). Apparently, in the absence of this protein, the mRNA level went up. Similarly, the inhibin alpha mRNA level increased in the presence of CHX, suggesting that both types of mRNAs are down regulated. However, the similarity ends when the mRNA levels were compared for granulosa cells that were treated with FSH for 24 hours. In these cells, the CHX effects were opposite on the mRNA levels of inhibin alpha and TM4, substantially suppressing the former whereas slightly enhancing the latter (FIG. 8). These opposite effects are of interest because the two gene transcripts showed the second peak at 24 hours of exposure to FSH (FIG. 7).

The effects of FSH and forskolin that activate adenylyl cyclase were the same. These results suggest that FSH induces the expression of the cytoskeletal and inhibin alpha genes via adenylyl cyclase. This conclusion is consistent with the previous report that FSH induces the massive reorganization of the actin cytoskeleton through the adenylyl cyclase pathway. It should be noted that alpha amanitin blocked the FSH-induced mRNA expression of all of the cytoskeletal and inhibin alpha genes, suggesting transcriptional regulation.

Tissue Specific Expression of the Cytoskeletal Genes

FSH up-regulates the mRNA expression of the three cytoskeletal genes in the ovarian granulosa cells. To test whether this parallel up-regulation of the three genes is tissue specific, total RNAs were isolated from different tissues of adult female and male rats and analyzed. Although the ovary, uterus and lung noticeably expressed the three cytoskeletal genes, their relative expression levels were diverse, particularly in the brain, pancreas and testis (data not shown). The results suggest the tissue specificity of the three genes' coexpression in the granulosa cells.

It is demonstrated herein, (using several different methods: differential display, slot blot and Northern analysis), that three cytoskeletal genes, β-tubulin, TM4 and Khc, were rapidly up- and down-regulated in response to FSH in ROG cells; not only the mRNA levels but also the β-tubulin and TM4 proteins. Furthermore, these proteins were assembled into massive cytoskeletons during up-regulation. These results underscore their crucial roles in granulosa cell differentiation and follicular development. Equally significant is the fact that it occurred not only in ROG cells but also in vivo. For instance, the similar up- and down-regulation of the three cytoskeletal genes is demonstrated in freshly isolated granulosa cell cultures and developing follicles of the rat ovaries. Therefore, their gene expression is dependent not only on exposure to FSH but also on the exposure period of the hormone, as well as the stage of follicular development.

The quick increase and peaking in their mRNA levels after FSH treatment are demonstrated by several lines of evidence, Northern blots and RT-PCR analyses, as well as in situ hybridization studies of both randomly cycling adult rats and immature rats treated for superovulation. This temporal expression of TM4 and β-tubulin mRNAs is confined in the large preantral and early antral stages (FIGS. 5 and 6). These results suggest an immediate functional role of the cytoskeletal genes in the FSH-induced granulosa cell differentiation and proliferation.

The cytoskeleton is involved in a variety of cellular functions including intracellular communication, cell polarity, locomotion, establishment and maintenance of morphology, cell to cell and cell to substratum contacts, and cell division. There are three major classes of cytoskeletal structures, actin microfilaments, microtubules and intermediate filaments. Tropomyosin binds as head to tail aggregates in a groove along the f-actin helix thus strengthening the filament, and has been intensively studied in muscle cells, where it regulates the binding of myosin heads to f-actin. However, in non-muscle cells, tropomyosin is known to have roles in intracellular granule movement, vesicular transport, mRNA localization and mitosis. TM4 is one of 18 distinct soforms generated by alternative splicing and alternative promotor usages, whose specific function is unknown. Polymers of tubulin make up microtubules, which are important in a wide range of cellular processes, including intracellular transport and generation of the mitotic spindle in most of cells. Kinesin, consisting of two heavy chains and two light chains, binds directly to microtubules and provides anterograde transport of vesicles and organelles.

The wide range of novel observations described herein is the first demonstration of the relationship of these gene transcripts and proteins and their FSH-responsive temporal expression to granulosa differentiation and follicular development. These results are consistent with physiological observations. In cycling rats, a few primordial follicles are recruited into the growth cycle by the secondary FSH surge and, under FSH signaling, the granulosa cells of the selected follicles undergo morphological changes, differentiation and proliferation. These are accompanied by changes in the intracellular structure, organelle movement and steroidogenesis. Therefore, these dynamic changes likely require an immediate supply of specific cytoskeletal proteins, and one of the functions of FSH at this early stage of granulosa cell differentiation is to trigger the synthesis of these cytoskeletal proteins by inducing their mRNA expression. It is of interest that the basal mRNA levels of the cytoskeletal genes were notable in primary granulosa cells but not in ROG cells. In contrast, ROG cells were derived from a primordial follicle and had never been exposed to FSH. The quick surge in the three cytoskeletal genes' transcripts, followed by the secondary mRNA expression peaking at FSH exposure for 24-48 hours (FIG. 7), suggests a complex regulation of their gene expression.

Indeed, the FSH-dependent increase in the three genes' mRNAs is regulated by complex and distinct mechanisms, although FSH induced their transcription. For example, CHX blocked the six hours FSH induced expression of β-tubulin mRNAs, indicating the requirement of de novo protein synthesis. In contrast, CHX enhanced the FSH-induced TM4 mRNA level, suggesting the existence of a negative regulator protein such as RNase. Interestingly, the inhibin alpha gene responded likewise. On the other hand, the Khc mRNA level was insensitive to the agent. Therefore, although all three genes are up-regulated by FSH, upregulation for the three genes occurs by different mechanisms. This diverse transcriptional regulation further diverges at a later stage of differentiation. The effects of CHX are different when the cells are treated with FSH for 24 hours (FIG. 8), implying that FSH dependent gene expression is dependent on the differentiation stage. This is consistent with tissue specific expression of the genes. Since FSH and forskolin, an adenylyl cyclase activator, impacted these genes' expression in the same way, the cytoskeletal genes are regulated by FSH via the adenylyl cyclase/cAMP pathway.

In conclusion, three cytoskeletal genes, β-tubulin, TM4 and Khc, were identified as FSH responsive genes. They are transiently expressed in granulosa cells at the preantral and early antral stages of follicular development. The regulation of the expression of these genes can be manipulated by exposing the genes to FSH. Such exposure during the large preantral and early antral stages of follicular development alters folliclular development, granulosa cell differentiation, and/or microtubule formation.

### EXAMPLE 1

FSH and hCG were purchased from the National Hormone and Peptide Program. Dulbecco's modified Eagle's Media (DMEM), Hams-F12 and antibiotics for tissue culture were from Gibco-BRL (Gaithersburg, Md.) and all general reagents were from Sigma Chemical Co (St. Louis, Mo.). Restriction enzymes, reverse transcriptase, T3, T7 and SP6 RNA polymerases, and Taq DNA polymerase were obtained from New England Biolabs (Beverly, Mass.). [³⁵S]-UTP□S and [³²P]-dCTP were from Amersham Pharmacia Biotech (Piscataway, N.J.). Oligonucleotides were synthesized by Sigma. (Coralville, Iowa).

Animals, Hormone Treatment, Granulosa Cell Isolations and Culture

Eighteen to twenty-one day old Sprague-Dawley female pups with nursing mothers were purchased from Harlan Breeding Company (Indianapolis, Ind.) and housed in a photoperiod of 14 h light/10 h darkness with light on at 0500 h. For in situ hybridization, rats were injected subcuteneoulsy with 15 IU PMSG (Sigma) in 0.1 ml PBS on 22-23 days of age. Depending on the experiments, PMSG 48 hours-primed rats were injected i.p. with 10 IU hCG. Ovaries were frozen on dry ice immediately after excision and stored at −80 C. To harvest and culture granulosa cells exhibiting a small antral phenotype, immature rats were injected s.c. with 1.5 mg of 17□-estradiol once a day on days 21, 22, and 23. Ovaries were collected in ice-cold serum-free 4F medium consisting of 15 mM HEPES (pH 7.4), 50% DMEM and 50% Ham's F12 with bovine transferrin (5 micro g/ml), human insulin (2 mg/ml), hydrocortisone (40 ng/ml) and antibiotics, and processed as previously described. Cells were washed three times, resuspended in 4F, plated on serum-coated. Six-well plate (1×10⁶ cells in 2 ml per well), and incubated at 37 C for 16 hours. FSH (30 ng/ml) or forskolin (10 micro M) was added to the cell cultures. For the inhibition of protein synthesis or transcription, 10 micro g/ml of cycloheximide (CHX) or alpha-amanitin (30 microg/ml) was, respectively, added 1 h before hormone treatment.

ROG cells were cultured as previously described (Li et al., Endocrinology 138:2648-2657). Briefly, ROG cells were maintained in suspension in a defined serum free medium consisting of F12-Dulbecco's modified Eagle's medium (DMEM) supplemented with Activin A (25 ng/ml), insulin (10 μg/ml), transferrin (5 μg/ml), □-tocopherol (0.1 μg/ml), progesterone (10 nM), bovine serum albumin (0.1%), and aprotinin (25 μg/ml) in the absence of antibiotics. Activin A (25 ng/ml) was replenished every 24 h. The cells were provided with fresh media once a week, pooled every two weeks by centrifugation at 1000 rpm for 5 min and replated at 1:2.

Reverse Transcription-Polymerase Chain Reaction (RT-PCR)

RT-PCR was performed essentially as previously described (Ko, et al., Endocrinology 140:5185-5194. Total RNA (1-2 micro g) was reverse-transcribed at 37 C in a 20 micro L reaction volume using random hexamer (500 ng) and MMLV reverse transcriptase (10 units) (New England BioLabs, Boston, Mass.). Complementary DNA (cDNA) samples (2 micro L) were added to a total 25 micro L reaction mix containing the primers (200 ng each), 0.4 mM dNTP mixture, and Taq DNA polymerase (2.5 U) in 1×PCR buffer (10 mM Tris, pH 8.3, 50 mM KCl, 1.5 mM MgCl₂, 0.01% gelatin). All PCR amplifications were carried out for 20, 25 or 30 cycles on a MJ Research Minicycler. PCR products were separated by 2% agarose gel electrophoresis, stained with SYBR® Green I (Molecular Probes), and visualized by phosphoimaging technology (FLA-2000; Fuji, Stamford, Conn.). The following primers were used in the present studies: TM4 (5′-gag aac tcc tga ctg aac tgg acg-3′(SEQ ID NO. 1) and 5′-cca tat tcc ctg ctg agc gta g-3′(SEQ ID NO. 2), 282 bp), Khc (5′-aac tga atc gcc tcc aag cag-3′ and 5′-cga act ggc gag aac tgg atg-3′, 195 bp), β-tubulin (5′-cct gct cat cag caa gat tcg-3′(SEQ ID NO. 3) and 5′-gtg gtg agc tta agg gta cgg, (SEQ ID NOI 4) 210 bp), and inhibin alpha subunit (5′-gct ttc cct ctg ttg acc cac-3′ (SEQ ID NO. 5) and 5′-aga tgt tga ggg cag ctc gat-3′, (SEQ ID NO. 6) 255 bp). L-19 (5′-ctg aag gtc aaa ggg aat gtg-3 (SEQ ID NO. 7) and 5′-gga cag agt ctt gat gat ctc, (SEQ ID NO. 8) 194 bp) oligo nucleotide primers were used to amplify ribosomal protein L-19 as an internal control.

Differential Display

ROG cells were incubated in the absence or presence of FSH (30 ng/ml) for 6 hours in triplicate prior to extracting total RNA. Total RNA was used as a template for differential display analyses using the Delta™ Differential Display Kit (Clonetech Laboratories, Inc., Palo Alto, Calif.). Briefly, total RNA was reverse-transcribed to produce first-strand cDNA by incubating 2 μg total RNA with an oligo (dT)₁₂₋₁₈ primer and MMLV reverse transcriptase for 1 hour at 42 C. Reactions were performed in triplicate and the cDNAs pooled to reduce variability. The pooled first-strand cDNA was diluted by transferring 2 μl to a new tube and adding 78 μl of sterile water (tube B), and then 72 μl sterile water to the original tube (tube A). Thus, each sample is run in duplicate but at different dilutions, reducing variability and false positives. PCR amplification of duplicate cDNA samples was performed in a MJ Research MiniCycler using a P/T primer pair in the presence of [³²P]dATP and Advantage KlenTaq polymerase (Clontech Laboratories). The following amplification program was used: 1 cycle of 95 C for 5 min, 40 C for 5 min, 68 C for 5 min; 2 cycles of 94 C for 2 min, 40 C for 5 min, 68 C for 5 min; 25 cycles of 94 C for 1 min, 60 C for 1 min, 68 C for 2 min and 1 cycle of 68 C for 10 min. Samples were electrophoresed on denaturing 5% polyacrylamide/8M urea gels. The gels were dried and exposed to X-ray film overnight at −80 C. Lanes containing vehicle and FSH samples were compared and bands appearing to be differentially regulated in both duplicate lanes were excised from the dried gel and eluted in 40 μl sterile water at 100 C for 5 min. The fragments were then re-amplified using the same P/T primer combination and the following amplification profile: 1 cycle of 95 C for 5 min, 20 cycles of 94 C for 1 min, 60 C for 1 min and 68 C for 2 min, and 1 cycle of 68 C for 10 min. Fragments were then sub-cloned into the pCR2.1 TA cloning vector system (Invitrogen Corporation, Carlsbad Calif.). Differential regulation of the subclones was verified by cDNA blot and/or Northern blot as described below. Each verified subclone was sequenced on a Beckman CEQ XL2000 capillary sequencer. Clones were identified using the BLAST algorithm at the National Center for Biotechnology Information (NCBI) (26).

Northern Blot

For Northern analysis, 10 μg of total RNA per sample was resolved on 1.5% agarose gels containing 2.2 M formaldehyde. RNA was then capillary-blotted to nylon membranes (Nytran super charge, Schleicher & Schuell Keene, NH). [□-³²P]dCTP-labeled cDNA probes were made from each subclone using the rediprime™ II random prime labeling system (Amersham Pharmacia Biotech, Piscataway, N.J.) and purified with micro bio-spin chromatography columns (Bio-Rad laboratories, Hercules, Calif.). Blots were hybridized overnight at 42 C in 50% (v/v) formamide, 5×SSPE, 5× Denhardt's reagent, 0.1% (w/v) SDS, and 200 mg/ml denatured, fragmented herring testis DNA. Filters were washed once at low stringency (5×SSPE, 1% SDS, 25 C) and twice at high stringency (0.1×SSPE, 1% SDS, 62 C) for 45 min and exposed to Kodak X-AR film for 12-72 hours at −80 C.

cDNA Blots

Differentially regulated cDNA fragments cloned into the pCR2.1 TA vector were amplified using M13 reverse and T7 primers. The samples were extracted once with phenol:chloroform and precipitated by adding 3M NaOAC, pH 5.2 (1:0.1) and 100% ETOH (1:2.5) and centrifuging at 14000 g at 4 degrees C. Pellets were resuspended in TE (pH 8). Each re-amplified fragment was denatured by adding 0.1 volume 3M NaOH and incubating for 1 h at 70 degrees C. Samples were cooled to room temperature and 20×SSPE (3.6M NaCl, 0.2M NaPO₄ pH 7.7, 20 mM EDTA) was added to a final concentration of 6×. Samples were immobilized on nylon membranes (Nytran super charge) using a Minifold II slot-blotter (Schleicher & Schuell). Each sample was divided equally among the appropriate number of filter membranes depending on experiment size. To probe the slot blots, first strand cDNA was synthesized from 5 μg total RNA using SuperScript™ II (Life Technologies, Grand Island N.Y.) and labeled with [□³²P]dCTP using the rediprime™ II random prime labeling system. Probes were purified with micro bio-spin chromatography. The slot-blots were hybridized overnight in the presence of probe at 42 C in 50% (v/v) formamide, 5×SSPE, 5× Denhardt's reagent, 0.1% (w/v) SDS, and 100 μg/ml denatured, fragmented herring testis DNA. Filters were washed once at low stringency (5×SSPE, 1% SDS, 25 C) and twice at high stringency (0.1×SSPE, 1% SDS, 62 C) for 45 min and exposed onto Kodak X-AR film for 12-72 h at −80 degrees C. Autoradiograms were digitized using an AGFA SNAPSCAN 1212 flatbed scanner and Adobe PhotoShop 5 software. Images were quantitated using Image J software (written by Wayne Rasband at the U.S. National Institutes of Health and available by anonymous FTP from zippy.nimh.nih.gov) and percent increase calculated for three independent experiments.

In Situ Hybridization

Frozen ovaries were cut in 20 micrometer sections using a MICROM HM 505 E cryostat (Microm Labogerate GmbH, Germany) and mounted onto Superfrost/Plus Microscope slides (Fisher Scientific, PA). Sections were fixed, pre-treated, and hybridized with antisense and sense RNA probes as previously described. Using T7, T3 or SP6 polymerase, [³⁵S]-UTP-labeled RNA probes were synthesized from the clone JK#7 for tropomyosin-4, JK#25 for Kinesin heavy chain, JK#46 for β-tubulin and the clone JK#56 for inhibin alpha, which have been subcloned into pBluescript II vector (Stratagene). RNA probes (1×10⁷ cpm/ml in hybridization buffer: 50% formamide, 5×SSPE, 2× Denhardt's reagent, 10% dextran sulfate, 0.1% SDS, and 100 micro g/ml yeast tRNA) were applied to sections and the sections were incubated in a humidity chamber at 47 C for 16-18 h. After hybridization, sections were treated with RNAse A (20 micro g/ml) at 37 degrees C. for 30 minutes, washed in decreasing concentrations of SSC down to 0.1×SSC at 58 C, and dehydrated through an ethanol series. Slides were then exposed to Kodak BIOMAX MR film for 2 days and processed for liquid emulsion autoradiography using NTB-2 emulsion (Kodak, Rochester, N.Y.) for 3-6 weeks. Developed sections were stained with Gill's Formulation #2 hematoxylin solution (Fisher Scientific). Tissues were examined with a Nikon Microphot-SA microscope (Nikon, Melville, NY) under bright- and dark-field optics. For each gene, a sense riboprobe was used as a control for nonspecific binding.

Immunofluorescent Staining

ROG cells were cultured on 12 mm number 1 glass coverslips in 24 well plates and allowed to attach overnight. Before plating, cover slips were coated with 10 μg/ml poly-D-lysine and 5 μg/ml fibronectin to facilitate cell attachment as per manufacturer's instruction (Sigma, St. Louis). Under these conditions, cell aggregates will attach loosely to the surface. After allowing cells to attach overnight, the medium was removed and the cells were incubated in defined media with or without 30 ng/ml FSH prior to being fixed and stained. Cells stained for f-actin were washed once in PBS (phosphate buffered saline), fixed in 4% paraformaldahyde in PBS at room temperature for 10 min, and permeablized in 0.1% triton X 100 for 5 minutes. Cells were then washed three times in PBS and incubated with PBSBT (1% bovine serum albumin and 0.05% Tween-20 in PBS) for 10 min. F-actin was stained by incubating slides with 0.7 μg/ml TRITC-phalloidin (Sigma, St. Louis) for 20 minutes.

Cells stained for tropomyosin or alpha-tubulin antibodies were fixed in −20 degrees C. 100% methanol at room temperature for 5 minutes and air dried. Cells were then washed three times in PBS and incubated with PBSB (1% bovine serum albumin/PBS) for 10 min. Tubulin or tropomyosin was stained by incubating cells with alpha-tubulin antibody (1:200) or tropomyosin antibody (1:100) (Sigma, St. Louis) diluted in PBSB for one hour. Coverslips were washed three times with PBSB followed by incubation with an anti-mouse FITC conjugate (1:100) (Sigma) for 20 minutes. The fluorescence-labeled samples were viewed using a Lieca scanning confocal microscope with a krypton-argon laser. Projections were constructed using the NIH image software (written by Wayne Rasband at the U.S. National Institutes of Health and available by anonymous FTP from zippy.nimh.nih.gov) and pasted into Photoshop 5.0.

What is claimed is:

1. A method of altering the regulation of expression of a gene selected from Class I β-tubulin, tropomyosin-4, kenesin heavy chain and combinations thereof comprising contacting the gene with an effective amount of follicle stimulating hormone (FSH).

2. The method of claim 1 wherein the gene is contained within a granulosa cell.

3. The method of claim 2 wherein the granulosa cell is contacted with FSH at the preantral or early antral stage of follicle development.

4. A method of altering regulation of follicular development, atrophy and/or ovulation comprising exposing immature granulosa cells to an effective amount of follicle stimulating hormone at an early stage of granulosa cell differentiation.

5. A method of altering differentiation of immature granulosa cells comprising exposing the immature granulosa cells to an effective amount of follicle stimulating hormone at an early stage of granulosa cell differentiation.

6. A method of altering beta-tubulin gene expression during follicular development comprising exposing the large preantral and early antral follicules of an animal to an effective amount of follicle stimulating hormone at an early stage of immature granulosa cell differentiation.
